(19) United States
(12) Patent Application Publication
(10) Pub. No.: US 2005/0131505 A1
(43) Pub. Date: Jun. 16, 2005

(54) Lead insertion support device

(75) Inventor: Masayoshi Yokoyama, Suginami-ku (JP)

Correspondence Address: JORDAN AND HAMBURG LLP, 122 EAST 42ND STREET, SUITE 4000, NEW YORK, NY, 10168, US

(73) Assignee: Japan General Medical Institute Co., Ltd., Suginami-ku (JP)

(21) Appl. No.: 10/731,981

(22) Filed: Dec. 10, 2003

(51) Int. Cl. A61N 1/05

(52) U.S. Cl. 607/116

(57) ABSTRACT

The lead insertion support device is comprised of an insertion support part with a pointed end so as to be inserted the tip part inserted into a vein without damaging the wall of the blood vessel; and an insertion support device body with elasticity and flexibility further including a connection part attached to the insertion support part; a support part provided so as to project to the connection part perpendicularly; and a holding part attached to the tip portion of the support part, capable of be held with a finger. Therefore, it can inert the lead into a blood vessel quickly and safely even though the user is not skilled, and it can smoothly insert the lead into the blood vessel.

### BACKGROUND OF THE INVENTION

This invention relates to a lead insertion support device which is used when the lead used for a pacemaker is inserted into a blood vessel.

In the conventional insertion method for the lead, there are the phlebotomy (venisection) and the venipuncture method.

In the conventional venipimeture method as the lead insertion method, the blood vessel is damaged, the puncture device punctures the wall of arty and it bleeds badly, and the electrode breakage after an operation may develop complications.

Moreover, since the operation takes time and the person is not skilled if it is hard technically in the phlebotomy (venisection), and it is forced a burden great to a patient, and since the tip part of support device body (vein lifter) as the lead insertion support device is especially formed in the shape of a sharpened triangle, the wall of the vein is damaged, and it is easy to make a hole.

### SUMMARY OF THE INVENTION

Accordingly, in the phlebotomy (venisection), it is an object of the invention to provide a lead insertion support device that can insert the lead into a blood vessel quickly and safely even though the user is not skilled. It is another object of the invention to provide a lead insertion support device that can smoothly insert the lead into the blood vessel.

Novel features which are believed to be characteristic of the invention, both as to its organization and method of operation, together with further objects and advantages thereof, are described below with reference to the accompanying drawings in which preferred embodiments of the invention are illustrated as an example.

It is to be expressly understood, however, that the drawings are for the purpose of illustration and description only, and are not intended as a definition of the limits of the invention.

### BRIEF DESCRIPTION OF THE DRAWINGS

FIG. 1 is a front view showing a first embodiment of the present invention;

FIG. 2 is a back view showing a first embodiment of the present invention;

FIG. 3 is a side view showing a first embodiment of the present invention;

FIG. 4 is a plan view showing a first embodiment of the present invention;

FIG. 5 is a bottom view showing a first embodiment of the present invention;

FIG. 6 is an explanation view showing the way in which an insertion support device is inserted;

FIG. 7 is an explanation view showing the way in which a first lead is inserted;

FIG. 8 is an explanation view showing the way in which a second lead is inserted;

FIG. 9 is a reference view in side;

FIG. 10 is a reference view in plan;

FIG. 11 is a front view showing a second embodiment of the present invention;

FIG. 12 is a side view showing a second embodiment of the present invention;

FIG. 13 is a front view showing a third embodiment of the present invention;

FIG. 14 is a side view showing a third embodiment of the present invention;

FIG. 15 is a cross sectional view taken along the line 15-15 in FIG. 13;

FIG. 16 is a front view showing a fourth embodiment of the present invention;

FIG. 17 is a side view showing a fourth embodiment of the present invention;

FIG. 18 is a front view showing a fifth embodiment of the present invention;

FIG. 19 is a side view showing a fifth embodiment of the present invention;

FIG. 20 is a plan view showing a fifth embodiment of the present invention;

FIG. 21 is a front view showing a sixth embodiment of the present invention;

FIG. 22 is a side view showing a sixth embodiment of the present invention;

FIG. 23 is a plan view showing a sixth embodiment of the present invention;

FIG. 24 is an explanation view when in use;

FIG. 25 is a front view showing a seventh embodiment of the present invention;

FIG. 26 is a side view showing a seventh embodiment of the present invention;

FIG. 27 is a plan view showing a seventh embodiment of the present invention;

FIG. 28 is a front view showing an eighth embodiment of the present invention;

FIG. 29 is a side view showing an eighth embodiment of the present invention; and

FIG. 30 is a plan view showing an eighth embodiment of the present invention.

### DETAILED DESCRIPTION OF THE PREFERRED EMBODIMENTS

Preferred embodiments of the present invention are described in more detail below with reference to the accompanying drawings.

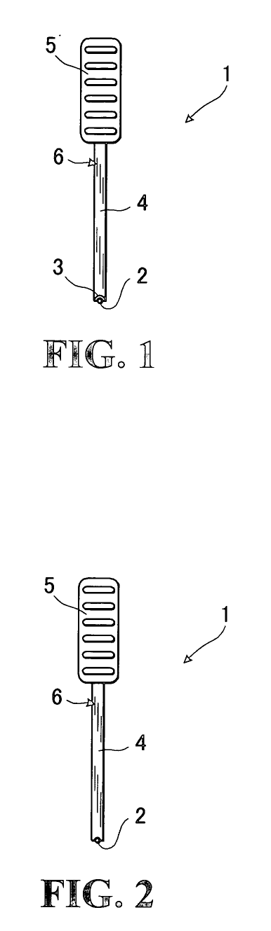

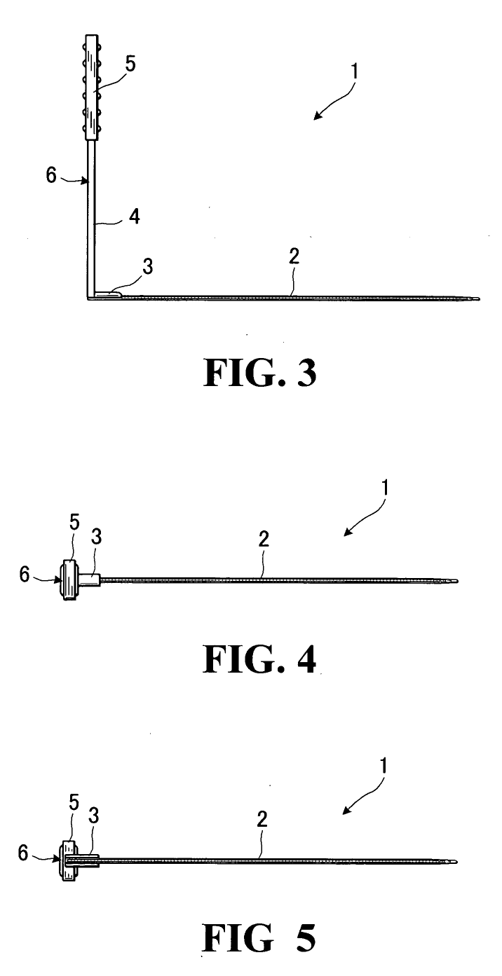

FIGS. 1-10 illustrate a lead insertion support device in accordance with a first embodiment of the present invention.

The numeral 1 shows a lead insertion support device which can insert the lead for a pacemaker in the blood vessel, especially vein. The lead insertion support device 1 is composed of an insertion support part 2 with elasticity and flexibility, tip portion of thereof forming in the shape of a pointed end so as to be inserted the tip part inserted into a vein without damaging the wall of the blood vessel; and an insertion support device body 6, which is made from synthetic resin material, with elasticity and flexibility, further including a connection part 3 attached fixedly the insertion support part 2, a support part 4, which is formed in the shape of a rod, provided so as to project to the connection part 3 perpendicularly and a holding part 5 attached to the tip portion of the support part 4, capable of be held with a finger.

The insertion support part 2 is formed with the same material as the conventional lead, and it is formed in the length of approximately 3-10 cm, preferably 6-10 cm so that it can insert without damaging the wall of the blood vessel in this embodiment.

In addition, although the insertion support part 2 is formed with the same material as the conventional lead, since it may be formed with the wire etc. with flexibility, and the insertion support part 2 may be covered by some covers with an antibacterial protection because it is inserted into the blood vessel.

In the holding part 5 of the insertion support device body 6 in the present invention, the concavo-convex shape is formed at the surface of the holding part 5 in order not to slide the finger during work, and it may be designed the shape that the finger is not slide during work.

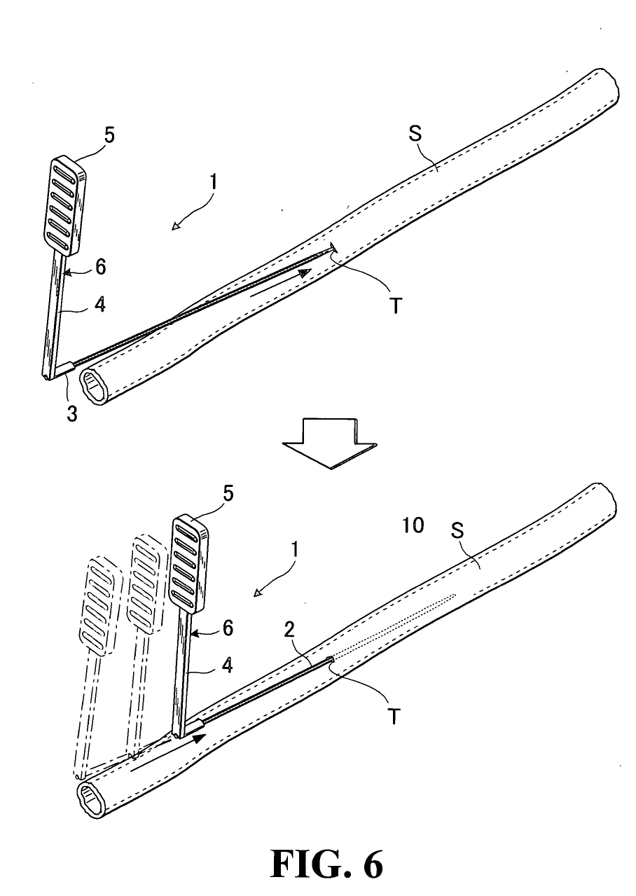

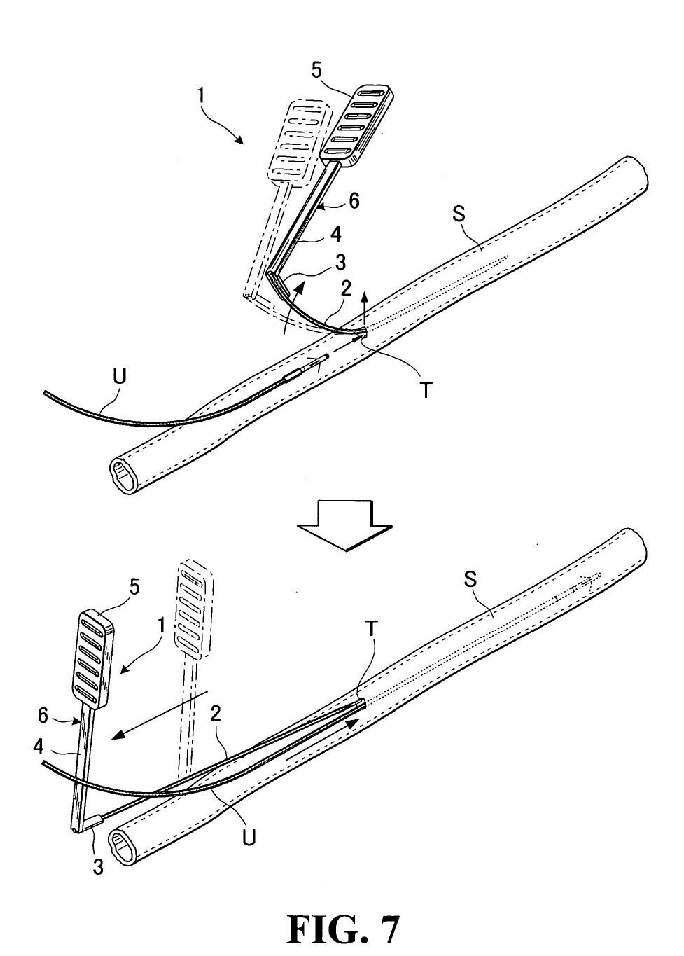

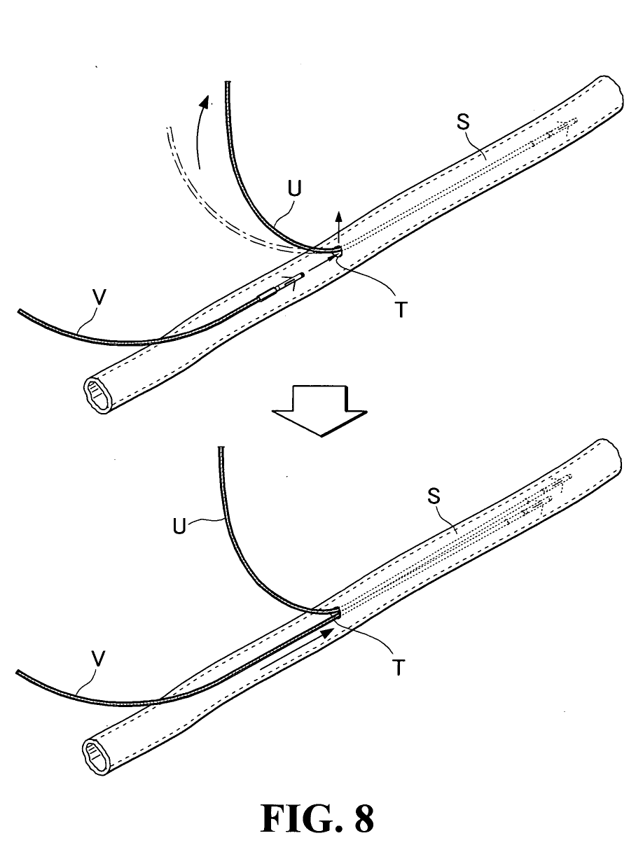

In the phlebotomy (venisection) used in the lead insertion support device 1, the insertion, as shown in FIG. 6, the insertion support part 2 is inserted in an insertion hole T which is cut and opened on the vein S. As shown in FIG. 7, after that, the holding part 5 or the support part 4 is held with the finger, and the insertion hole T is extended in the case of raising upwards so that the first lead U is inserted into the extended insertion hole T of the vein S. After the insertion of the first lead U is checked, the insertion support part 2 is extracted from the vein S. Then, the first lead U has an insertion state. Furthermore, as shown in FIG. 8, the first lead U is raised, and the second lead V is inserted into the insertion hole T of the vein S.

Therefore, since the tip part of the insertion support part 2 is mostly formed in the shape of a tip and there is a certain amount of length, the insertion support part 2 can be inserted smoothly along with the blood vessel without damaging the wall of the blood vessel. Furthermore, since the first lead U is smoothly inserted along with the insertion support part 2 so that it may be introduced to the insertion support part 2 tat is inserted into the blood vessel, the insertion work can be done quickly.

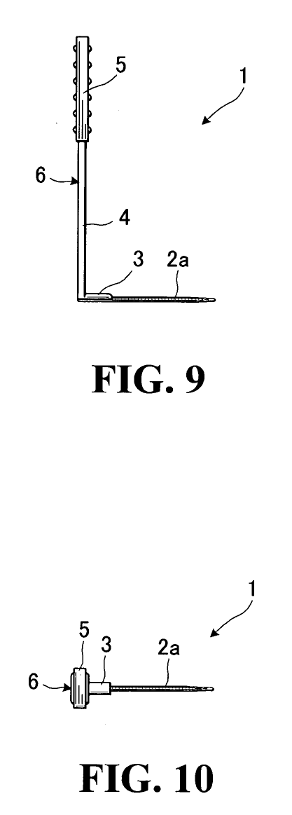

In addition, as shown in FIG. 9 and FIG. O, the insertion support part 2a that is shorter than the insertion support device body 6 may be attached.

Furthermore, in the embodiment, the pacemaker with two leads is explained, also the pacemaker with one lead is operated as well as two leads.

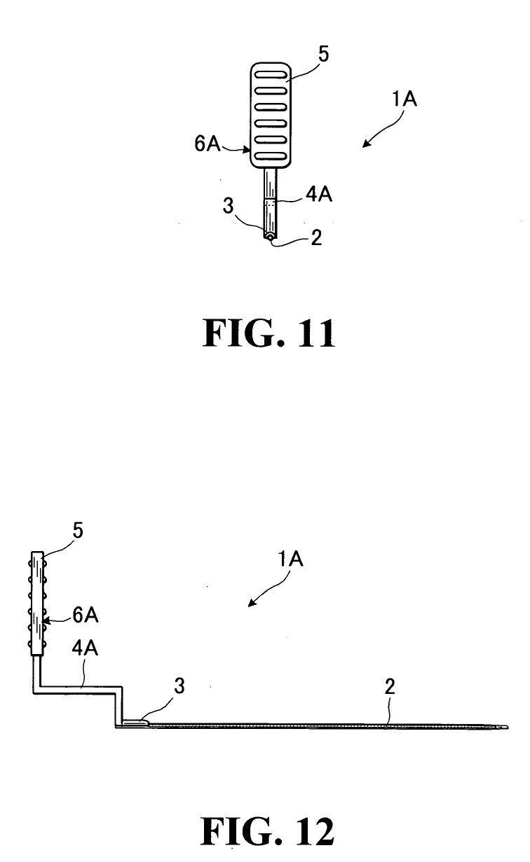

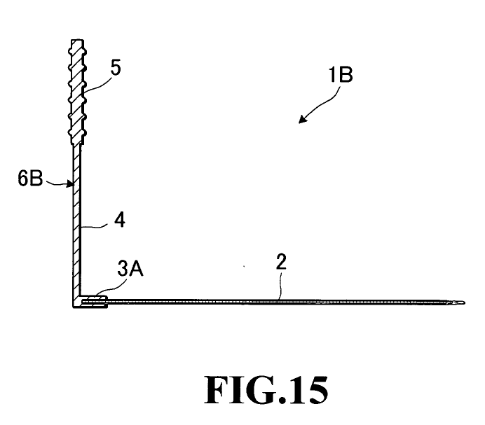

Other embodiments of the present invention will now be described with reference to FIGS. 11-30. In FIGS. 11-30, the same components as in the first embodiment described above with reference to FIGS. 11-30 are designated by the same reference numerals and therefore will not be further explained in great detail.

A second embodiment of the present invention is shown in FIGS. 11 and 12. It is distinguished from the first embodiment in that the support part 4 is replaced from another support part 4A which is formed in the shape of a crank. A lead insertion support device 1A with an insertion support device body 6A according to the second embodiment has similar advantages to that according to the first embodiment.

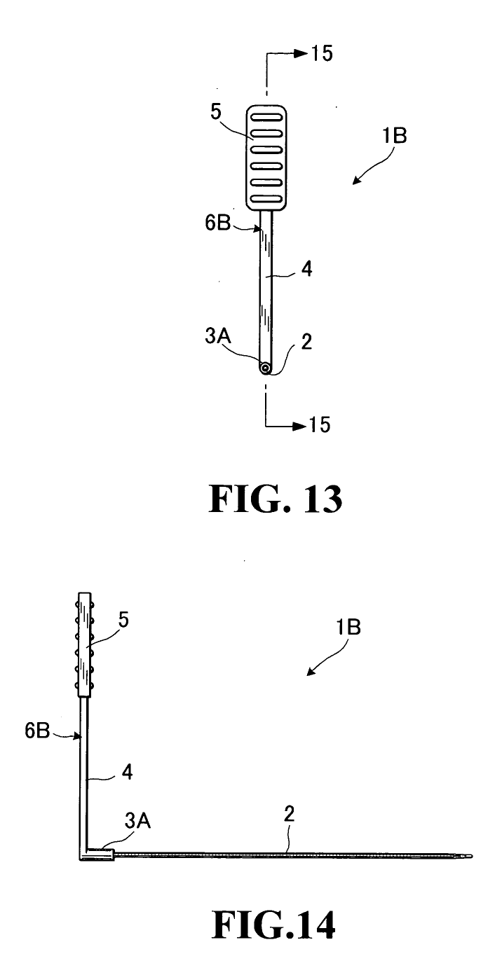

A third embodiment of the present invention is shown in FIGS. 13 to 15. It is distinguished from the first embodiment in that the connection part 3 is replaced from another connection part 3A which is formed in the shape of a sleeve so as to be attached fixedly in the state that the insertion support part 2 is inserted thereinto. A lead insertion support device 1B with an insertion support device body 6B according to the third embodiment has similar advantages to that according to the first embodiment.

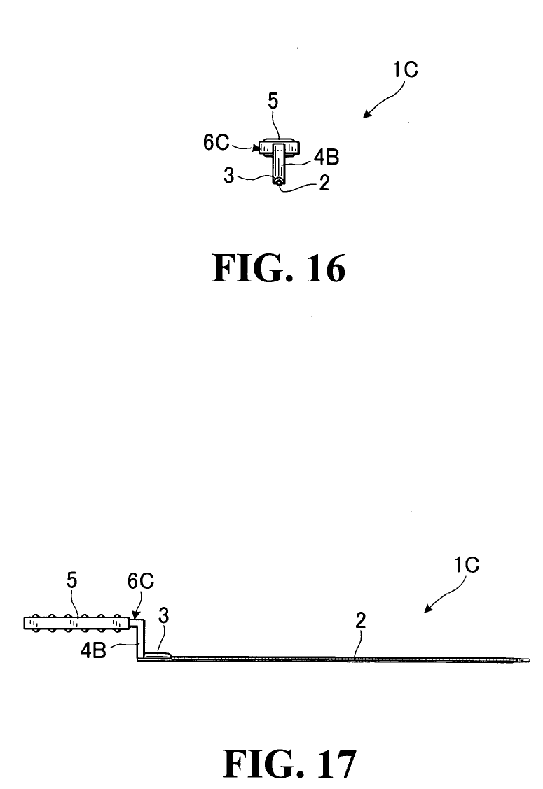

A fourth embodiment of the present invention is shown in FIGS. 16 and 17. It is distinguished from the first embodiment in that the support part 4 is replaced from another support part 4B which is formed in the shape of an angle. A lead insertion support device 1C with an insertion support device body 6C according to the fourth embodiment has similar advantages to that according to the first embodiment.

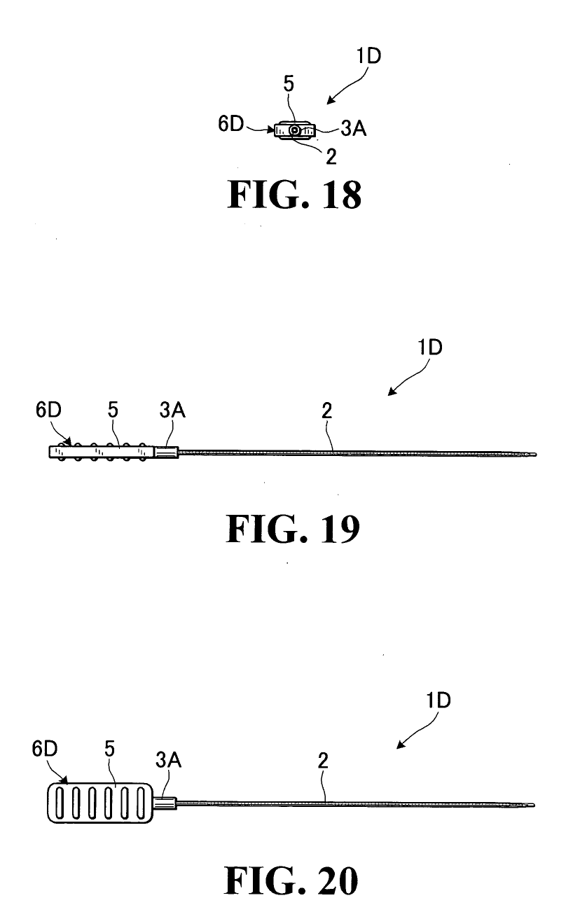

A fifth embodiment of the present invention is shown in FIGS. 18 to 20. It is distinguished from the first embodiment in that the connection part 3 is replaced from another connection part 3A attached fixedly the insertion support part 2, and the insertion support device body 6 is replaced from another insertion support device body 6D which is attached to a tip portion of the connection part 3A, having the holding part 5. A lead insertion support device 1D with the insertion support device body 6D according to the fifth embodiment has similar advantages to that according to the first embodiment.

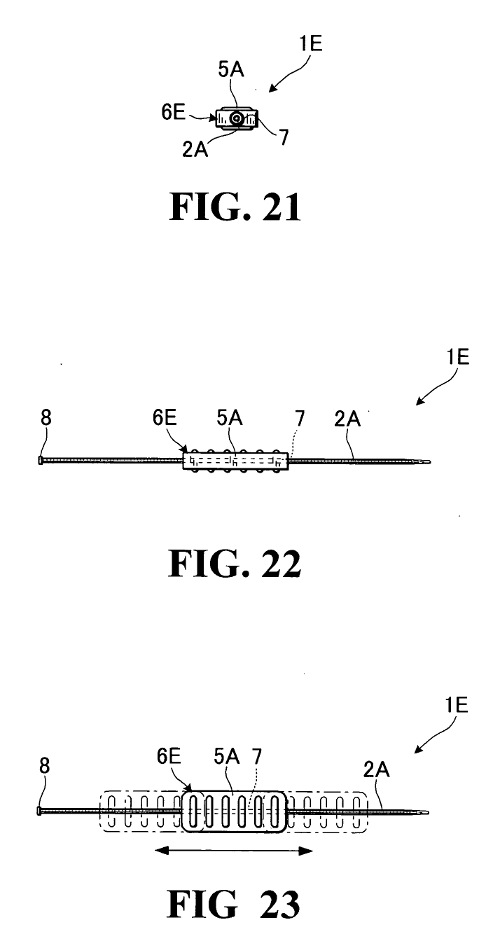

A sixth embodiment of the present invention is shown in FIGS. 21 to 24. It is distinguished from the first embodiment in that the holding part 5 is replaced from another holding part 5A capable of holding by a finger, having a through hole 7 formed at approximately central portion thereof, capable of moving slidely so as to hold the insertion support part 2 at any positions, and the insertion support part 2 is replaced from another insertion support part 2A which has a stopper 8 attached at an end thereof. A lead insertion support device 1E with an insertion support device body 6E according to the fifth embodiment has similar advantages to that according to the first embodiment.

In this embodiment, since the holding part 5A is formed by resin material which has pliability, the central part of the holding part 5A curves with an attached state with the insertion support part 2A when the user pushes strongly a central part of the holding part 5A and holds fixedly. Therefore, the user holds certainly the holding part 5A in any positions on the insertion support part 2.

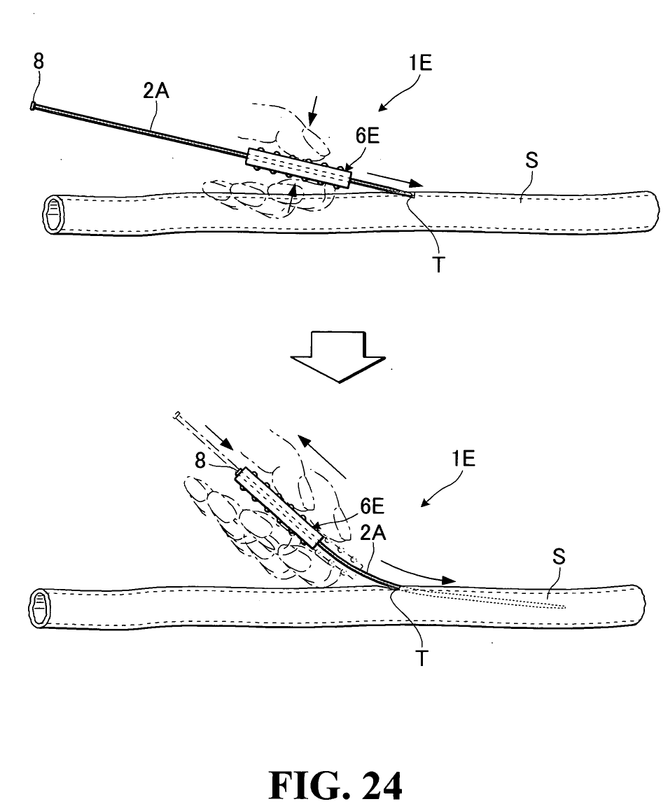

Therefore, as shown in FIG. 24, when the insertion support part 2A is inserted into the insertion hole T of the vein S, the holding part 5A is allowed to move slidely to the direction of the tip end of the insertion support part 2A, and the user holds the holding part 5A. Then the user can insert the insertion support part 2A into the insertion hole T.

After that, when the first lead U is inserted into the vein S, the holding part 5A is allowed to move slidely to the direction of the back end of the insertion support part 2A, and the user holds the holding part 5A. Then, the pacemaker lead U is inserted into the vein S certainly, and the user can remove the insertion support part 2A from the vein 2 after the insertion state is checked.

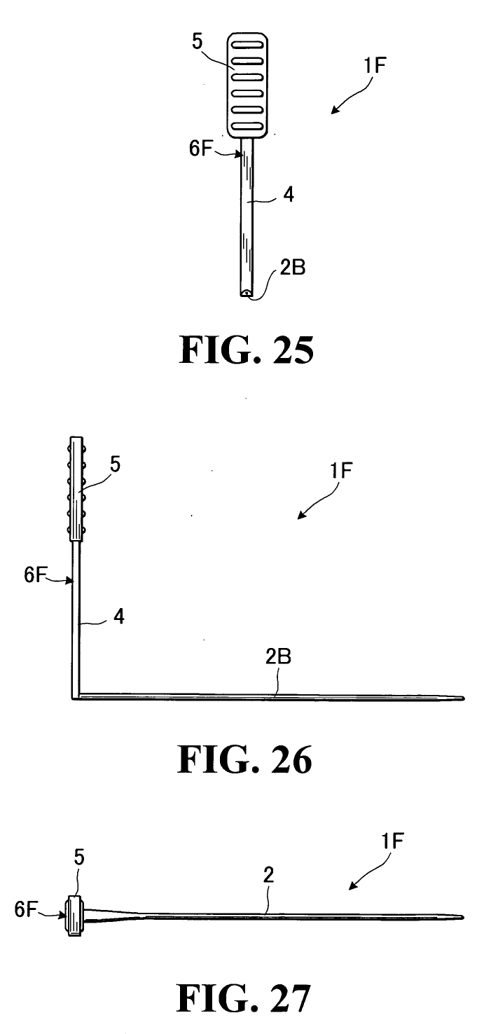

A seventh embodiment of the present invention is shown in FIGS. 25 to 27. It is distinguished from the first embodiment in that the insertion support device body 6 is replaced from another insertion support device body 6F which is formed an insertion support part 2B with elasticity and flexibility integrally. A lead insertion support device 1F with the insertion support device body 6F according to the seventh embodiment has similar advantages to that according to the first embodiment.

In addition, in the second, fourth and fifth embodiments, the insertion support part 2B may be formed integrally with the insertion support device body 6A, 6C and 6D.

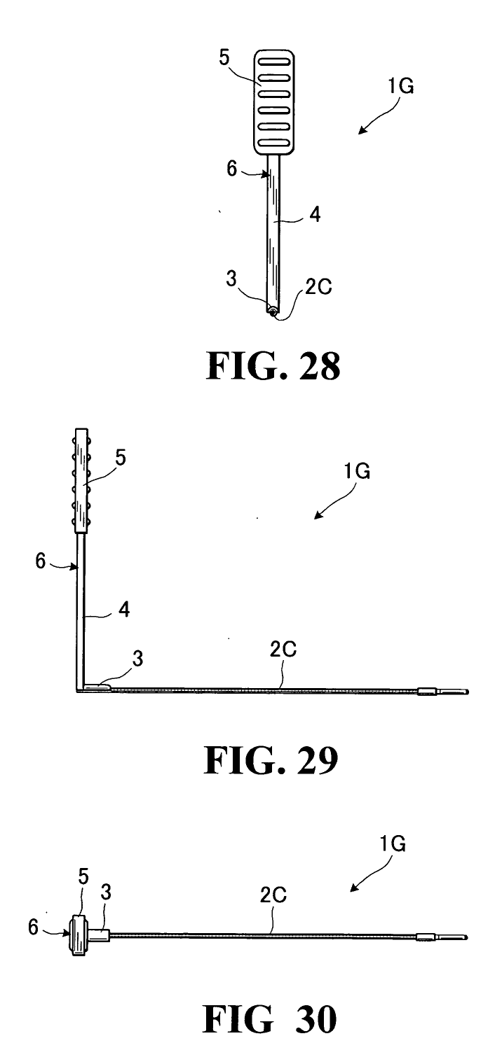

An eighth embodiment of the present invention is shown in FIGS. 28 to 30. It is distinguished from the first embodiment in that the insertion support part 2 is replaced from another insertion support part 2C as a lead without the hook (tine) of the conventional pacemaker. A lead insertion support device 1G with the insertion support part 2C according to the eighth embodiment has similar advantages to that according to the first embodiment.

In each embodiment, the lead insertion support device used when the pacemaker lead used for the peacemaker is inserted into the blood vessel is explained, also it may be used in order to insert the lead which uses in order to induce discharge of chemistry substances such as serotonin and dopamine after an electric pulse is sent through a cerebral neuron.

As set forth above, the advantages of the invention are as follows:

(1) A lead insertion support device is comprised of an insertion support part with elasticity and flexibility, a tip portion thereof forming in the shape of a pointed end so as to be inserted the tip part inserted into a vein without damaging the wall of the blood vessel; and an insertion support device body, which is made from synthetic resin material, with elasticity and flexibility, further including a connection part attached one of fixedly and integrally to the insertion support part; a support par, which is formed in the shape of a rod, provided so as to project to the connection part perpendicularly; and a holding part attached to the tip portion of the support part capable of be held with a finger. Therefore, the insertion hole can be extended certainly without the damage of the wall of the blood vessel.

Therefore, the lead of the pacemaker can be inserted along with the blood vessel smoothly.

(2) As discussed above, since the tip part of the insertion support part 2 is mostly formed in the shape of a tip and there is a certain amount of length; the insertion support part can be inserted smoothly along with the blood vessel without damaging the wall of the blood vessel.

(3) As discussed above, the insertion hole can be extended and managed easily without damaging the wall of the blood vessel so that the lead can be inserted into the blood vessel quickly and safely even though the user is not skilled.

(4) As discussed above, it has simple structure so that the cost can be reduced

1. A lead insertion support device comprising:
an insertion support part with elasticity and flexibility, a tip portion thereof forming in the shape of a pointed end so as to be inserted the tip part inserted into a vein without damaging the wall of the blood vessel; and
an insertion support device body, which is made from synthetic resin material, with elasticity and flexibility, further including a connection part attached one of fixedly and integrally to the insertion support part; a support part which is formed in the shape of a rod, provided so as to project to the connection part perpendicularly; and a holding part attached to the tip portion of the support part, capable of be held with a finger.

2. A lead insertion support device according to claim 1 wherein the insertion support part has a tip portion thereof forming in the shape of a pointed end so as to be ins the tip part inserted into a vein without damaging the wall of the blood vessel, and it is formed in the length of approximately 3-10 cm.

3. A lead insertion support device according to claim 1 wherein the support part is formed in the shape of one of a crank and an angle.

4. A lead insertion support device according to claim 1 wherein the insertion support device body includes the connection part attached one of fixedly and integrally to the insertion support part and the holding part attached to the tip portion of the connection part, capable of be held with a finger.

5. A lead insertion support device according to claim 1 wherein an insertion support part which has a stopper attached at an end thereof; and an insertion support device body with a holding part, capable of holding by a finger, having a through hole formed at approximately central portion thereof, capable of moving slidely so as to hold the insertion support part at any positions.
